(19) United States
(12) Patent Application Publication
(10) Pub. No.: US 2026/0327720 A1
(43) Pub. Date: Oct. 22, 2026

(54) METHODS AND COMPOSITIONS USEFUL IN DISCRIMINATING BETWEEN FISH SPECIES

(71) Applicant: The Florida State University Research Foundation, Inc., Tallahassee, FL (US)

(72) Inventor: Prashant Singh, Tallahassee, FL (US)

(21) Appl. No.: 19/712,504

(22) Filed: Jun. 18, 2026

Related U.S. Application Data
(63) Continuation of application No. 18/341,277, filed on Jun. 26, 2023.
(60) Provisional application No. 63/390,321, filed on Jul. 19, 2022.

(51) Int. Cl. C12Q 1/6888

(52) U.S. Cl. CPC C12Q 1/6888; C12Q 2600/156; C12Q 2600/158; C12Q 2600/16

(57) ABSTRACT

Determining whether a particular sample is what it is labeled or sold as is an important concept, particularly to those who buy or sell food products. Particularly for end users, a fast, efficient, accurate way of determining whether a sample is being accurately marketed and sold is necessary. This invention allows a user to rapidly determine if a product is from a certain species or not, based on genetic markers for that product.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Jun. 26, 2023, is entitled “10850-068US1_ST26.xml”, and is 16,744 bytes in size.

### CROSS-REFERENCE TO RELATED APPLICATIONS

This application claims benefit of U.S. Provisional Application No. 63/390,321, filed Jul. 19, 2022, incorporated herein by reference in its entirety.

### BACKGROUND

The gold standard method for authenticating the identity of seafood species is the FDA's DNA barcoding technique, which targets the cytochrome C oxidase subunit I (COI) gene of the fish (Handy et al., 2011). Two barcoding methods are used for the identification of seafood specimens. The method targeting the ~650 bp region is called barcoding and is applicable for fresh or minimally processed seafood specimens. Whereas the method targeting a smaller ~100-300 bp region is referred to as mini-barcoding and is often useful for processed seafood specimens (Isaacs et al., 2020). The resulting sequence data for the sample is compared with FDA standard barcodes (Kress and Erickson, 2012). Sample sequence showing greater than 98% similarity with a standard barcode sequence is considered a positive match. The barcoding method is a robust method for seafood species identification and can identify up to 2749 species (FDA, 2011). However, as the process involves the overnight shipment of samples to a testing laboratory, DNA isolation, PCR amplification, sample clean up, sequencing of samples at a core facility, and data analysis the whole process can take up to five days. This lengthy testing time is a problem which limits the use of technology for seafood species identification.

Rapid seafood species-specific PCR-based tests that eliminate the need for DNA barcoding have been developed (Bayha et al., 2018; Lee et al., 2021; Wilwet et al., 2017; Isaacs et al., 2020). These assays commonly target the COI or 16S rRNA gene sequence for assay development. However, many species used for the substitution have a target gene sequence, which differs by only a few bases making the assay prone to false-positive results.

There is a need in the art for a low-cost, rapid assay to discern whether a certain sample is a same seafood species as mentioned on the label or not. Furthermore, there is a need for this method that can be performed onsite, at an in-house food processing facility, in a resource limited setting, using a minimally trained labor.

### SUMMARY

The present invention relates to a method of rapidly determining if a specific food product is present or not, the method comprising: providing a sample comprising at least one target sequence; placing the sample into at least one container; using reagents to amplify a sample; amplifying a sample using a small footprint nucleic acid amplification device, wherein the sample is amplified by exposing it to different sets and types of primers (conventional and rhPCR primers) in conditions suitable for nucleic acid amplification, where each set of primers comprises of a forward and reverse primers; exposing the amplified sequence to a means of detection, wherein the means of detection provides a present/not present result; and identifying whether the food or ingredient is present or not, based on the results.

Also disclosed is a kit can comprise a container for amplification of a sample; an instrument for sample collection; reagents for amplification of the sample; an instrument for rapid amplification of the sample; and a means of detecting whether the specific food product is present or not.

Further disclosed are nucleic acids with 90% or more identity to SEQ ID NOS: 1, 2, 3, 4, 5, and/or 6, or any combination thereof.

Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description are exemplary and explanatory only and are not restrictive of the disclosure.

### BRIEF DESCRIPTION OF THE FIGURES

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIG. 1A-D shows an assay overview: A) is fish meat, B) is a toothpick used to scrape a sample from the fish, C) is a typical reaction tube, D) is a Watson PCR machine (IEH Laboratories & Consulting Group, Seattle, WA), which is used to process the assay.

FIG. 1E shows a overall assay workflow standardized for the seafood testing.

FIG. 2A-B shows PCR-Lateral Flow assay. A) PCR-lateral flow assay for the specific identification of white shrimp. B) PCR-lateral flow assay for the specific identification of white shrimp with an internal amplification control (IAC).

FIG. 3A-B shows PCR-Lateral Flow assay for the identification of red snapper samples. A) Barcoded samples showing positive identification for the red snapper. B) Barcoded samples showing negative test results for other fin fish species.

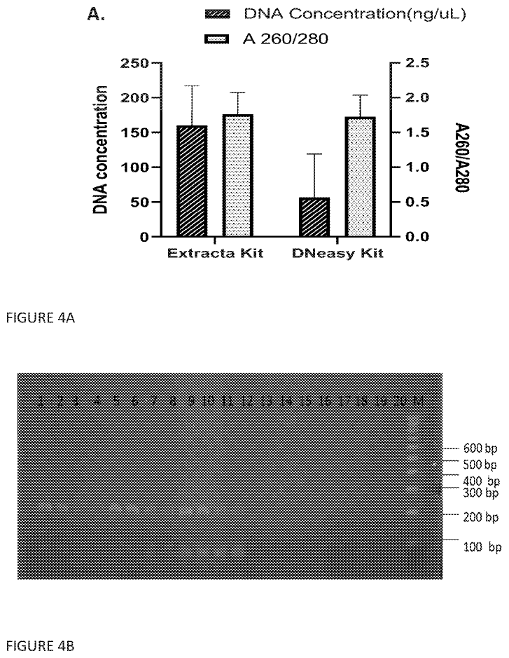

FIG. 4A-D shows a comparison of DNA extraction from snapper and non-snapper samples. FIG. 4A: Comparison of DNA extraction yield from two kits. FIG. 4B: Lane 1-12 showing a 160 bp red snapper specific amplicon with red snapper specific rhPCR primers. Lane 13-20 are non-red snapper samples, Lane M—DNA marker. FIG. 4C: Lane 1-12 showing a 600 bp IAC amplicon for the red snapper samples. Lane 13-20 are non-red snapper samples. FIG. 4D: Strip 1—Lateral flow test to confirm test band for red snapper primer. Strip 2—lateral flow test to confirm test band for IAC primer.

### DETAILED DESCRIPTION

### Definitions

In this specification and in the claims which follow, reference will be made to a number of terms which shall be defined to have the following meanings:

As used herein, the singular forms “a,” “an” and “the” include plural referents unless the context clearly dictates otherwise. Thus, for example, reference to a “metal” includes examples having two or more such “metals” unless the context clearly indicates otherwise.

Ranges can be expressed herein as from “about” one particular value, and/or to “about” another particular value. When such a range is expressed, another example includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

As used herein, “complementary” or “complementarity” refers to the ability of a nucleotide in a polynucleotide molecule to form a base pair with another nucleotide in a second polynucleotide molecule. For example, the sequence 5′-A-C-T-3′ is complementary to the sequence 3′-T-G-A-5′. Complementarity may be partial, in which only some of the nucleotides match according to base pairing, or complete, where all the nucleotides match according to base pairing. For purposes of the present invention “substantially complementary” refers to 90% or greater identity over the length of the target base pair region. The complementarity can also be 50, 60, 70, 75, 80, 85, 90, 91, 92, 93, 94, 95, 96, 97, 98, 99, or 100% complementary, or any amount below or in between these amounts.

As used herein, “nucleic acid sequence” refers to the order or sequence of nucleotides along a strand of nucleic acids. In some cases, the order of these nucleotides may determine the order of the amino acids along a corresponding polypeptide chain. The nucleic acid sequence thus codes for the amino acid sequence. The nucleic acid sequence may be single-stranded or double-stranded, as specified, or contain portions of both double-stranded and single-stranded sequences. The nucleic acid sequence may be composed of DNA, both genomic and cDNA, RNA, or a hybrid, where the sequence comprises any combination of deoxyribo- and ribo-nucleotides, and any combination of bases, including uracil (U), adenine (A), thymine (T), cytosine (C), guanine (G), inosine, xathanine hypoxathanine, isocytosine, isoguanine, etc. It may include modified bases, including locked nucleic acids, peptide nucleic acids and others known to those skilled in the art.

An “oligonucleotide” is a polymer comprising two or more nucleotides. The polymer can additionally comprise non-nucleotide elements such as labels, quenchers, blocking groups, or the like. The nucleotides of the oligonucleotide can be natural or non-natural and can be unsubstituted, unmodified, substituted or modified. The nucleotides can be linked by phosphodiester bonds, or by phosphorothioate linkages, methylphosphonate linkages, boranophosphate linkages, or the like.

A “primer” is a nucleic acid that contains a sequence complementary to a region of a template nucleic acid strand and that primes the synthesis of a strand complementary to the template (or a portion thereof). Primers are typically 18-20 base long, but need not be, relatively short, chemically synthesized oligonucleotides (typically, deoxyribonucleotides). In an amplification, e.g., a PCR amplification, a pair of primers typically define the 5′ ends of the two complementary strands of the nucleic acid target that is amplified.

By “capture sequence,” which is also referred to herein as a “second nucleic acid sequence” is meant a sequence which hybridizes to the target nucleic acid and allows the first nucleic acid sequence, or primer sequence, to be in close proximity to the target region of the target nucleic acid.

A “target region” is a region of a target nucleic acid that is to be amplified, detected or both.

The “Tm” (melting temperature) of a nucleic acid duplex under specified conditions is the temperature at which half of the nucleic acid sequences are disassociated and half are associated. As used herein, “isolated Tm” refers to the individual melting temperature of either the first or second nucleic acid sequence in the cooperative nucleic acid when not in the cooperative pair. “Effective Tm” refers to the resulting melting temperature of either the first or second nucleic acid when linked together.

As used herein, “amplify, amplifying, amplifies, amplified, amplification” refers to the creation of one or more identical or complementary copies of the target DNA. The copies may be single stranded or double stranded. Amplification can be part of a number of processes such as extension of a primer, reverse transcription, polymerase chain reaction, isothermal polymerase chain reaction, nucleic acid sequencing, rolling circle amplification and the like.

As used herein, “purified” refers to a polynucleotide, for example a target nucleic acid sequence, that has been separated from cellular debris, for example, high molecular weight DNA, RNA and protein. This would include an isolated RNA sample that would be separated from cellular debris, including DNA. It can also mean non-native, or non-naturally occurring nucleic acid.

As used herein, “protein,” “peptide,” and “polypeptide” are used interchangeably to denote an amino acid polymer or a set of two or more interacting or bound amino acid polymers.

As used herein, “stringency” refers to the conditions, i.e., temperature, ionic strength, solvents, and the like, under which hybridization between polynucleotides occurs. Hybridization being the process that occurs between the primer and template DNA during the annealing step of the amplification process.

As used herein, “multiplex” refers to the use of PCR to amplify several different DNA targets (genes) simultaneously in a single assay or reaction. Multiplexing can amplify nucleic acid samples, such as genomic DNA, cDNA, RNA, etc., using multiple primers and any necessary reagents or components in a thermal cycler.

As used herein, a “sample” is from any source, including, but not limited to, a gas sample, a fluid sample, a solid sample, or any mixture thereof. In a preferred embodiment, the sample can be from fish, and can include, but is not limited to, scales, tissue, such as muscle or other flesh, or organs.

The term “sensitivity” refers to a measure of the proportion of actual positives which are correctly identified as such.

The term “confidence level” refers to the likelihood, expressed as a percentage, that the results of a test are real and repeatable, and not random. Confidence levels are used to indicate the reliability of an estimate and can be calculated by a variety of methods.

In certain embodiments, sequences of the present invention, including primer sequences, target sequences and IAC sequences may be identical to the sequences provided here in or comprise less than 100% sequence identity to the sequences provided herein. For instance, primer sequences, target sequences or IAC sequences of the present invention may comprise 90-100% identity to the sequences provided herein.

The terms “identical” or “percent identity,” in the context of two or more nucleic acids or sequences, refer to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., the NCBI web site found at ncbi.nlm.nih.gov/BLAST/ or the like). Such sequences are then referred to as “substantially identical.” This definition also refers to, or applies to, the compliment of a particular sequence. The definition may also include sequences that have deletions, additions, and/or substitutions. To compensate for gene sequence diversity and to target multiple gene variants of the same genes, degenerated primer pairs (1-2 bases or approximately 5-10% alterations) are allowed.

As used herein, the term “nucleic acid” refers to a single or double-stranded polymer of deoxyribonucleotide bases or ribonucleotide bases read from the 5′ to the 3′ end, which may include genomic DNA, target sequences, primer sequences, or the like. In accordance with the invention, a “nucleic acid” may refer to any DNA or nucleic acid to be used in an assay as described herein, which may be isolated or extracted from a biological sample. The term “nucleotide sequence” or “nucleic acid sequence” refers to both the sense and antisense strands of a nucleic acid as either individual single strands or in the duplex. The terms “nucleic acid segment,” “nucleotide sequence segment,” or more generally, “segment,” will be understood by those in the art as a functional term that includes genomic sequences, target sequences, operon sequences, and smaller engineered nucleotide sequences that express or may be adapted to express, proteins, polypeptides or peptides. The nomenclature used herein is that required by Title 37 of the United States Code of Federal Regulations § 1.822 and set forth in the tables in WIPO Standard ST.25 (1998), Appendix 2, Tables 1 and 3.

The term “gene” refers to components that comprise bacterial DNA or RNA, cDNA, artificial bacterial DNA polynucleotide, or other DNA that encodes a bacterial peptide, bacterial polypeptide, bacterial protein, or bacterial RNA transcript molecule, introns and/or exons where appropriate, and the genetic elements that may flank the coding sequence that are involved in the regulation of expression, such as, promoter regions, 5′ leader regions, 3′ untranslated region that may exist as native genes or transgenes in a bacterial genome. The gene or a fragment thereof can be subjected to polynucleotide sequencing methods that determines the order of the nucleotides that comprise the gene. Polynucleotides as described herein may be complementary to all or a portion of a bacterial gene sequence, including a promoter, coding sequence, 5′ untranslated region, and 3′ untranslated region. Nucleotides may be referred to by their commonly accepted single-letter codes.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has” or “includes” one or more steps is not limited to possessing only those one or more steps and also covers other unlisted steps. Similarly, any cell that “comprises,” “has” or “includes” one or more traits is not limited to possessing only those one or more traits and covers other unlisted traits.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular electrode is disclosed and discussed and a number of modifications that can be made to the electrode are discussed, specifically contemplated is each and every combination and permutation of the electrode and the modifications that are possible unless specifically indicated to the contrary. Thus, if a class of electrodes A, B, and C are disclosed as well as a class of electrodes D, E, and F and an example of a combination electrode, or, for example, a combination electrode comprising A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that the compositions disclosed herein have certain functions. Disclosed herein are certain structural requirements for performing the disclosed functions, and it is understood that there are a variety of structures that can perform the same function which are related to the disclosed structures, and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to the arrangement of steps or operational flow; plain meaning derived from grammatical organization or punctuation; and the number or type of embodiments described in the specification.

### General Description

Seafood is a substantial source of nutrients such as proteins, polyunsaturated fatty acids, vitamin A, iodine, zinc, calcium, iron, and ω-3 fatty acids, making them an important component of the human diet (Aaker et al., 2020, Liu et al., 2021). Worldwide, fish and fish products contribute to about 20% of the per capita animal protein intake (FAO, 2018). Between 2008 and 2018, the global seafood consumption rate increased by about 19%, from 16.7 kg per capita to 20.5 kg per capita (FAO, 2020b). In the United States (U.S.), fresh and frozen finfish consumption is about 3.8 kg (8.3 pounds) per capita (NMFS, 2022).

The northern red snapper (Lutjanus campechanus) is harvested from the Gulf of Mexico, and the southern red snapper (Lutjanus purpureus) is harvested from Brazil and the South Atlantic Ocean (Gomes et al. 2008). According to the National Oceanic and Atmospheric Administration (NOAA), the commercial landings of red snapper in 2021 were about 7.7 million pounds, with a worth of $33 million, which makes it one of the most commercially prominent seafood species in the United States (NOAA, 2022). However, due to a history of overfishing, red snapper stocks at catchment areas are depleting. These actions have resulted in the implementation of strict recreational and commercial harvesting limits by the NOAA Fisheries and Gulf of Mexico and South Atlantic Fisheries Management Councils (NOAA, 2022).

Due to high demand, commercial importance, and close morphological similarity with other fish species, red snapper is vulnerable to food fraud (Isaacs et al., 2020). Cases of these frauds are further exacerbated by the trading of fish in processed forms (e.g., fish filets), making them morphologically indistinguishable from other fish species (Hu et al., 2018). Food frauds such as species substitution and mislabeling are a global food safety concern and violate the Federal Food, Drug, and Cosmetic Act, Section 403 (FDA, 2009).

Between 2010-2012, Oceana conducted a study on the identification of retail fish samples. They collected samples from 21 states and verified the identity of samples using a DNA-based method. Results from the study revealed that about one-third of seafood samples were mislabeled. Furthermore, samples sold as snapper had the highest rate of mislabeling, with red snapper samples accounting for 93% of total snapper mislabeling (Warner et al., 2013). A similar observation was made by Spencer and Bruno (2019), where about 77% of red snappers collected from sushi restaurants, seafood markets, and grocery stores from the southeastern part of the United States were found to be misrepresented.

Fish commonly used to substitute red snapper in the United States include the cheaper alternative such as tilefish (Oreochromis niloticus), which contains considerable amounts of mercury and could lead to toxicological health risks when consumed (Warner et al., 2012). Additionally, the substitution of red snapper with tilapia can expose the consumers to zoonotic parasites such as Haplorchis spp., Gnathostoma spp., and Metagonimus spp., which are prominent in freshwater-farmed fish (Williams et al., 2020).

The gold standard method for authenticating the identity of seafood species is the FDA's DNA barcoding technique, which targets the cytochrome C oxidase subunit I (COI) gene of the fish (Handy et al., 2011). Two barcoding methods are used for the identification of seafood specimens. The method targeting the ~650 bp region is called barcoding and is applicable for fresh or minimally processed seafood specimens. Whereas the method targeting a smaller ~100-300 bp region is referred to as mini-barcoding and is often useful for processed seafood specimens (Isaacs et al., 2020). The resulting sequence data for the sample is compared with FDA standard barcodes (Kress and Erickson, 2012). Sample sequence showing greater than 98% similarity with a standard barcode sequence is considered a positive match. The barcoding method is a robust method for seafood species identification and can identify up to 2749 species (FDA, 2011). However, as the process involves the overnight shipment of samples to a testing laboratory, DNA isolation, PCR amplification, sample clean up, sequencing of samples at a core facility, and data analysis the whole process can take up to five days. This lengthy testing time is a problem which limits the use of technology for seafood species identification.

Rapid seafood species-specific PCR-based tests that eliminate the need for DNA barcoding have been developed (Bayha et al., 2018; Lee et al., 2021; Wilwet et al., 2017; Isaacs et al., 2020). These assays commonly target the COI or 16S rRNA gene sequence for assay development. However, many species used for the substitution have a target gene sequence, which differs by only a few bases making the assay prone to false-positive results. These limitations of PCR assay can be overcome using RNase H2-dependent PCR (rhPCR) approach (Dobosy et al., 2011). The rhPCR approach relies on the use of blocked primers and RNase H2 enzyme isolated from Pyrococcus abyssi (Will, 1992). RNase H2 enzyme enables the PCR assay to make a sequence-specific cut and activates the blocked primer only in the presence of the target DNA sequence. The rhPCR assay is highly specific toward their targets and can be performed using a conventional PCR instrument. Then those rhPCR amplicons can be detected using a lateral-flow kit. Therefore, the aim of this study was to standardize a highly specific rhPCR-coupled lateral flow assay, which can be used for the onsite identification of red snapper samples in a resource-limited setting.

### Methods of Detection

Disclosed herein is a method of rapidly determining if a specific food product is present or not, the method comprising: providing a sample comprising at least one target sequence; placing the sample into at least one container; using reagents to amplify a sample; amplifying a sample using a small footprint amplification device, wherein the sample is amplified by exposing it to different sets of primers in conditions suitable for nucleic acid amplification, where each set of primers comprises of a forward and reverse primer; exposing the amplified sequence to a means of detection, wherein the means of detection provides a present/not present result; and identifying whether the food product or ingredient is present or not based on the results.

The food product can be a fruit, vegetable, grain, or legume. The food product can also be beef, pork, poultry, fish, or shellfish or an allergen. In a preferred embodiment, the food product is seafood. Examples of seafood include, but are not limited to, edible fish such as American shad, American sole, anchovy, antarctic cod, arrowtooth eel, asian carp, atka mackerel, atlantic cod, atlantic eel, atlantic herring, atlantic salmon, atlantic trout, australasian salmon, black mackerel, blue cod, bluefin tuna, brook trout, butterfish, barramundi, California halibut, capelin, carp, catfish, cherry salmon, chinook salmon, chum salmon, cod, coho salmon, eel, european eel, european flounder, flathead, flatfish, flounder, freshwater eel, freshwater herring, groundfish, haddock, halibut, harvest fish, herring, hilsa, Japanese butterfish, John Dory, kapenta, lemon sole, mackerel, maori cod, mahi-mahi, milkfish, monkfish, northern anchovy, Norwegian Atlantic salmon, orange roughy, pacific cod, pacific herring, pacific salmon, pacific saury, pacific trout, panfish, pelagic cod, pink salmon, pollock, pilchard, rainbow trout, redfish, red snapper, round herring, Russian sturgeon (including eggs), salmon, sardine, saury, scrod, sea bass, seer fish, shrimp, silver carp, skipjack tuna, sole, snook, snoek, Spanish mackerel, sturgeon (including eggs), surf sardine, swamp-eel, swordfish, striped bass, skate, tilapia, trout, tuna, turbot, walleye, walu also known as butter fish, whitebait, whitefish, whiting, and yellowfin tuna.

Examples of seafood also include the edible eggs offish, such as caviar (sturgeon roe), Ikura (salmon roe), kazunoko (herring roe), lumpfish roe, masago (capelin roe), and tobiko (flying-fish roe). Examples of seafood also include shellfish, which includes molluscs and crustaceans such as crab, particularly dungeness crab, king crab, snow crab; crayfish; lobster, particularly American lobster and rock lobster/spiny lobster; shrimp; prawns; abalone; clam; cockle; conch; cuttlefish; mussel; octopus; oyster; periwinkle; snail; squid; and scallop, specifically bay scallop and sea scallop. Seafood also includes other aquatic organisms, such as sea cucumber and Uni (sea urchin “roe”).

In a specific example, the seafood is white shrimp (Litopenaeus setiferus) or red snapper (Lutjanus campechanus or Lutjanus purpureus).

The sample can be obtained from a variety of means. Typically, either a whole fish or shellfish can be sampled, or a piece of meat or other tissue sample can be used. A toothpick, tweezers, a swab, or other small device for gathering a DNA sample can be used. The device can be passed along the surface of the sample, or can be plunged into the sample. The device for gathering the sample can then be placed in a tube, where it can be processed through a DNA extraction procedures, and centrifuged if needed, although this is not a necessary step and samples can be directly processed using a inhibitor-resistant mater mix (i.e., Platinum Direct PCR Universal Master Mix, KAPA PROBE Force). In one embodiment, a commercially available kit, such as PrepMan® Ultra Sample Preparation Reagent (Applied Biosystems, Life Technologies) or Extracta DNA Prep for PCR (Quanta Biosciences, Beverly, MA, USA) may be used to isolate DNA. According to one embodiment, suspended food particles may be separated from the media, for instance through filtration or centrifugation of the enriched sample, for example at 3,000-10,000×g. The cell pellet can be heat treated at 95 °C for 10 to 30 minutes (depending upon the samples), samples can be centrifuged and obtained supernatant containing crude DNA extract can be used as a sample for analysis described herein. The sample can then be exposed to amplification reagents (known to those of skill in the art) and amplified.

Amplification can occur by using a variety of devices. In a preferred embodiment, the amplification device can be small footprint, portable device such as a Watson PCR machine. In some embodiments, the machine can weigh less than 5, 10, 15, or 20 lbs, and can be less than 12″×12″, 15″×15″, 18″×18″, or 24″×24″.

Once amplified, the sample can be placed on a detection device, such as a lateral flow assay (LFA). Such lateral flow assays for the detection of a sample are known to those of skill in the art. LFAs are typically composed of a nitrocellulose membrane, sample pad, conjugate pad, wicking or absorbent pad, and backing pad. Nitrocellulose membranes are most commonly used as they facilitate a support capable of use for both reaction and detection, with capture biomolecules e.g., antibodies, are deposited on the nitrocellulose to form the test and control lines via a combination of electrostatic interactions, hydrogen bonds and/or hydrophobic interactions (Jauset-Rubio et al., 2016), herein incorporated in its entirety for its teaching concerning lateral flow assays). There are a large number of paper analytical devices (PAD) that have been developed for detection of PCR products using lateral flow assays. There are two mains types of lateral flow nucleic acid tests, referred to as Nucleic Acid Lateral Flow (NALF) and Nucleic Acid Lateral Flow ImmunoAssay (NALFIA); NALF directly detects DNA exploiting capture and labeled reporter oligonucleotide probes, whereas NALFIA detects hapten-labeled DNA using capture and labeled reporter antibodies or streptavidin. Again, one of skill in the art can readily envision such assays for use with the present invention.

Importantly, the lateral flow assay can simply provide a “present/not present” result so that one skilled in the art can readily determine if the sample is a certain species or not. For example, if one were testing for the presence of red grouper, one would obtain a sample of meat, take a sample and amplify it, then expose the amplification product to a lateral flow assay designed to detect the presence of nucleic acid for red grouper. If red grouper is present, the assay will indicate a “positive.” Conversely, if red grouper is absent, the test will be negative, and no positive result will appear. In one embodiment, the lateral flow assay can comprise a control line to determine if the lateral flow strip and buffer are working or not. In one embodiment, the lateral flow assay can comprise an internal amplification control line to determine if PCR reactions are working or not. Again, one of skill in the art can readily determine how to design a lateral flow assay for use with this invention.

In some examples, a probe can be used to detect the target nucleic acid can be any probe known to those of skill in the art used in nucleic acid detection. The probe can be a single probe or a dual-labeled probe, such as those found in FRET systems. Detectable labels may include, but are not limited to, radiolabels, fluorochromes, including fluorescein isothiocyanate (FITC), biotin, digoxigenin, rhodamine, Texas Red, phycoerythrin, allophycocyanin, 6-carboxyfluorescein (6-FAM), 2′,7′-dimethoxy-4′,5′-dichloro-6-carboxyfluorescein, 6-carboxy-X-rhodamine (ROX), 6-carboxy-2′,4′,7′,4,7-hexachlorofluorescein (HEX), 5-carboxy fluorescein (5-FAM) or N,N,N′,N′-tetramethyl-6-carboxyrho-damine (TAMRA); radioactive labels such as 32P, 35S, and 3H), and the like. In some embodiments, a detectable label may involve multiple steps (e.g., biotin-avidin, hapten-anti-hapten antibody, and the like). A primer useful in accordance with the invention may be identical to a particular target nucleic acid sequence and different from other sequences.

The probes selected and/or utilized by the methodologies of the invention can provide sensitivity and/or specificity of more than 90%, 95%, 96%, 97%, 98%, 99% or 100%. In some embodiments, sensitivity and specificity depends on the hybridization signal strength, number of probes used, the number of potential cross-hybridization reactions, the signal strength of the mismatch probes, if present, background noise, or combinations thereof.

The oligonucleotide probes can each be from about 5 to about 100 nucleotides, from about 10 to about 50 nucleotides, from about 15 to about 35 nucleotides. or from about 20 to about 30 nucleotides. In some embodiments, the probes are at least 5-mers, 6-mers, 7-mers, 8-mers, 9-mers, 10-mers, 11-mers, 12-mers, 13-mers, 14-mers, 15-mers, 16-mers, 17-mers, 18-mers, 19-mers, 20-mers, 21-mers, 22-mers, 23-mers, 24-mers, 25-mers, 26-mers, 27-mers, 28-mers, 29-mers, 30-mers, 31-mers, 32-mers, 33-mers, 34-mers, 35-mers, 36-mers, 37-mers, 38-mers, 39-mers, 40-mers, 41-mers, 42-mers, 43-mers, 44-mers, 45-mers, 46-mers, 47-mers, 48-mers, 49-mers, 50-mers, 51-mers 52-mers, 53-mers, 54-mers, 55-mers, 56-mers, 57-mers, 58-mers, 59-mers, 60-mers, 61-mers, 62-mers, 63-mers, 64-mers, 65-mers, 66-mers, 67-mers, 68-mers, 69-mers, 70-mers, 71-mers, 72-mers, 73-mers, 74-mers, 75-mers, 76-mers, 77-mers, 78-mers, 79-mers, 80-mers, 81-mers, 82-mers, 83-mers, 84-mers, 85-mers, 86-mers, 87-mers, 88-mers, 89-mers, 90-mers, 91-mers, 92-mers, 93-mers, 94-mers, 95-mers, 96-mers, 97-mers, 98-mers, 99-mers, 100-mers or combinations thereof.

The amplification reaction described above needs reagents in order for amplification to occur. One of skill in the art can readily determine which reagents should be present in order to amplify a sample. Such reagents include, but are not limited to, PCR “Mastermix”; Taq polymerase; RNase H2 enzyme, RNase H2 enzyme buffer, and primers or labeled primers. Methods such as polymerase chain reaction (PCR, rhPCR, and RT-PCR) and ligase chain reaction (LCR) or isothermal PCR reaction may be used to amplify nucleic acid sequences directly from genomic material. For example, the PCR assay may include a number of reagents and components, including a master mix and nucleic acid dye or intercalating agent. In some embodiments, an exemplary PCR master mix may contain template genomic material, such as DNA or RNA, RNase H2 enzyme, RNase H2 enzyme buffer, PCR primers or labeled PCR primers, probes salts such as MgCl₂, a polymerase enzyme, and deoxyribonucleotides. One of skill in the art will be able to identify useful components of a master mix in accordance with the present invention.

### Specific Nucleic Acids

Disclosed herein are specific primers for amplifying the nucleic acid target from the sample. For example, when the target microorganism is white shrimp, the following primers can be used (sequences are found below in Example 1). One set of primers can comprise SEQ ID NOS: 1 and 2. A second set of primers can comprise SEQ ID NOS: 3 and 4. A third set of primers can comprise SEQ ID NOS: 5 and 6 or SEQ ID NOS: 7 and 8. Also disclosed are primers and probes comprising 80, 85, 90, 95, 96, 97, 98, or 99% or more identity to these primers and probes, or any amount above, below, or in between these amounts. Put another way, these primers and probes can have 1, 2, or 3 or more additions, deletions, or substitutions and are still contemplated for use with the methods and kits disclosed herein. Specifically disclosed herein are nucleic acids with 90% or more identity to any one of, or a combination of, SEQ ID NOS: 1, 2, 3, 4, 5, and 6.

### Kits

Also disclosed herein is a kit for amplification of nucleic acids. Kits may also include additional reagents, e.g., PCR components, such as salts including MgCl₂, a polymerase enzyme, and deoxyribonucleotides, and the like, reagents for DNA or RNA isolation, or enrichment of a biological sample, including for example media such as water, or the like, as described herein. Such reagents or components are well known in the art. Where appropriate, reagents included with such a kit may be provided either in the same container or media as the primer pair or multiple primer pairs, or may alternatively be placed in a second or additional distinct container into which the additional composition or reagents may be placed and suitably aliquoted. Alternatively, reagents may be provided in a single container means.

Specifically, the kit can comprise a container for amplification of a sample; an instrument for sample collection; reagents for amplification of the sample; an instrument for rapid amplification of the sample; and a means of detecting whether the specific food product is present or not. In some embodiments, the kit can further comprise a centrifugation means, as described above, such as a small footprint microcentrifuge. The sample collection instrument can be anything useful in collecting a sample, such as tweezers, a toothpick, or a swab, as described above. the PCR machine for rapid amplification can be a small footprint machine, such as a Watson PCR Machine. Further details are provided above. The reagents can include primers, such as the primers described herein.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is °C or is at ambient temperature, and pressure is at or near atmospheric. There are numerous variations and combinations of process conditions that can be used to optimize product quality and performance. Only reasonable and routine experimentation will be required to optimize such process conditions.

### Example 1: Lab-In-a-Suitcase Species Discrimination

Methods: Disclosed herein is a method that can be performed in resource-limited settings. The method relies on the following:
Species-specific SNP markers: Lutjanus peru, Lutjanus synagris, Lutjanus campechanus, and Lutjanus guttatus are morphologically and phylogenetically close. Therefore, conserved SNP markers are needed for the differentiation of closely related species. In-silico analysis has been performed to identify the conserved SNP marker to identify each species.
SNP-specific PCR amplification: As initially mentioned, the 500-600 bp COI gene sequence is commonly sequenced for the specific identification of seafood species. As the commercially important species and species used for misrepresentation are phylogenetically very close, it makes designing a species-specific PCR primer-pair challenging. This necessitates a need for a novel PCR approach that can be performed using a conventional PCR instrument and can be used by used by a minimally trained workforce.
RNase H-dependent PCR (rhPCR) assays rely on blocked rhPCR primers (rhPCR). The rhPCR primers are inactive due to a blocker molecule at the 3′-end. They can be specifically activated in the presence of specific SNP and RNase H2 enzyme. The RNase H2 enzyme only makes a cleavage when the RNA base of the rhPCR primers correctly bind to the target SNP. The cleaved rhPCR primers are active (possess a 3′-OH group) and can be amplified by Taq polymerase in a PCR reaction. Thus, facilitating a species-specific PCR amplification on a conventional PCR instrument.
PCR amplification from crude DNA extracts: DNA isolation can be challenging in a resource-limited setting due to a lack of laboratory equipment and a trained workforce. Further, a procedure that works with crude DNA extracts can help to save 1-2-hour sample processing time. The assay developed in this work relies on the use of an inhibitor-resistant Taq polymerase or master mix, which facilitates PCR amplification directly from crude extracts obtained after simple heat-lysis of the samples. Thus, eliminating the need for a complex DNA isolation process, simplifying the testing protocol, and making it suitable for onsite applications.
Portable PCR instrument: The food or seafood processing facilities rely on a small onsite quality control laboratory with limited space and equipment. The assay developed in this work has eliminated the use of a $2,000-5,000 big footprint conventional PCR or real-time PCR instruments and relies on the use of a low-cost portable PCR instrument ($200-300). Thus, making it suitable for onsite testing and adoption by industry processing facilities and quality control laboratories.
Visual detection of PCR amplicons: Agarose gel electrophoresis is a commonly used method for detecting PCR amplicons. However, the agarose gel electrophoresis is cumbersome, takes a long time, and requires specific equipment, gels, buffer, and dyes. Thus, they are not suitable for industrial processing facilities and quality control laboratories. The assay disclosed herein has combined rhPCR assay with lateral flow assay (i.e., single or two target). The lateral flow strips are premade, a highly sensitive method for detecting PCR amplicons, take 5-10 minutes, and it does not require any specific equipment.

Combined together, methods have been developed that can be used for onsite diagnostic applications, e.g., pathogen detection, seafood species identification, and allergen testing.

As a proof of concept, assays have been developed for the accurate identification of Atlantic white shrimp (Litopenaeus setiferus), and red snapper (Lutjanus campechanus). The developed assay was validated using a wide range of barcoded fish and shrimp samples.

Significance: The assay developed in this study for specific onsite identification top two seafood species that are commonly adulterated in the United States. Similar assays can be developed for other high-value species which are commonly misrepresented. The assay is simple and suitable for onsite deployment in a resource-limited setting and eliminates the need for expensive DNA barcoding-based seafood species identification, which can take 5-10 days. Further, the developed detection approach can be expanded for foodborne pathogens and allergen testing.

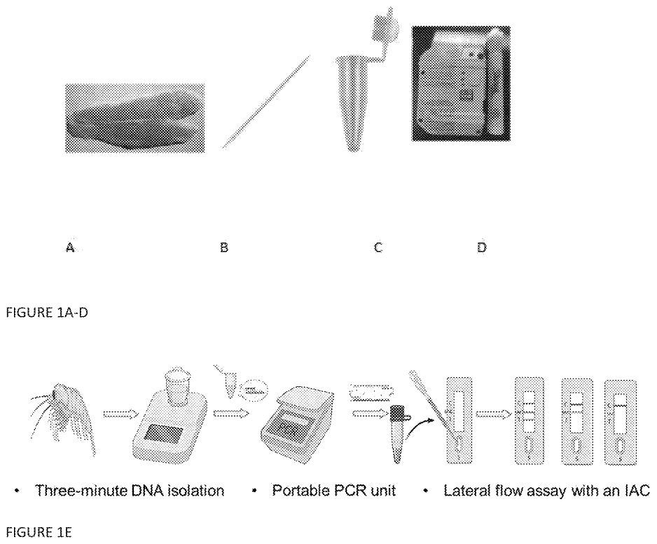

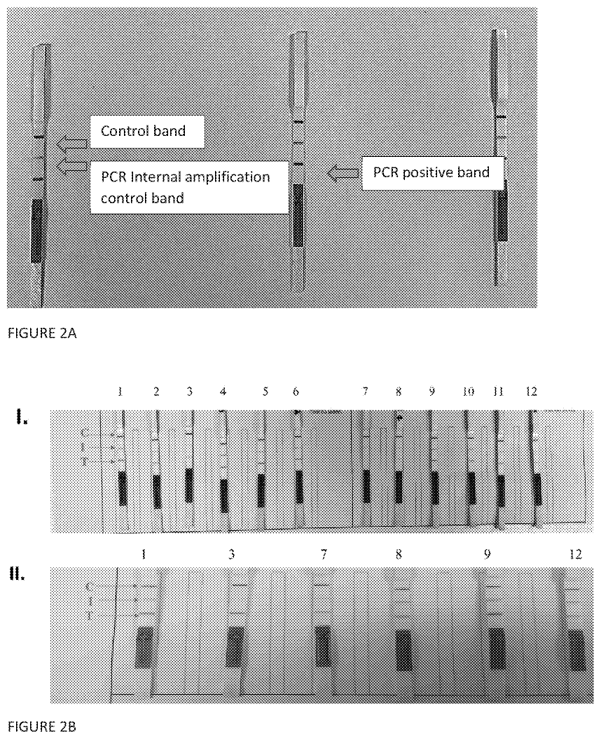

A lab in the suitcase can comprise the following:
Fine tweezers or other instrument: Used for collecting a very small amount of fish tissue sample.
Lyophilized PCR tubes: All the PCR reagents are lyophilized in the PCR tubes. For each target species, the suitcase has an individual PCR tube with specific reagents e.g., red snapper PCR tube, red grouper PCR tube, white shrimp PCR tube. The user can test the PCR tube based on the target species they intend to test.
PCR grade water: Used for activating the lyophilized reagent.
Microcentrifuge: A small footprint microcentrifuge is used to spin down the reagents before incubating in the PCR instruments.
Small PCR instrument: PCR assay is performed on a small footprint instrument (i.e., Watson PCR).
Lateral flow strips: The lateral flow kit is used for the confirmation of the presence and absence of PCR amplicons. The lateral flow is a commercially available product that makes the assay very reproducible.
Pipette: Two pipettes to add PCR grade water and samples.
(FIG. 1: Assay overview)
   Lateral Flow Assay for the Identification of Atlantic White Shrimp (Litopenaeus setiferus):
   Barcoded shrimp samples stored in the lab tissue collection was used for the standardization of the assay. The smallest amount of white shrimp sample, equivalent to a small grain of salt, was transferred to the PCR tube. The DNA from the tissue samples were isolated using a quick heat lysis step. White shrimp-specific primers labeled with FAM and biotin was used for the assay (Table 1). The PCR assay was performed directly from the crude DNA isolated from the shrimp tissue samples using the Platinum Direct PCR Universal Master Mix lysis reagent (Thermo Fisher Scientific). The tissue samples were heat lysed in the 20 μl lysis buffer at 98 °C for 1 min using the Watson PCR. Sample was centrifuged in a microcentrifuge, obtained supernatants were transferred to a new microcentrifuge tube, diluted 1/10 times to dilute the DNA concentrations, and 2 μl of diluted crude DNA extract was used for the PCR reaction. PCR was performed on a Watson PCR instrument. The white shrimp assay was performed using the primer (SEQ ID NO:1), (SEQ ID NO: 2), (SEQ ID NO: 3), and (SEQ ID NO: 4) in a multiplex format. In the PCR reaction, primer-pair (SEQ ID NO:1) and (SEQ ID NO: 2) specifically amplified white shrimp specific DNA sequence, whereas the primer-pair (SEQ ID NO:3) and (SEQ ID NO: 4) amplified a conserved 16S RNA region among various shrimp species and acted as an internal amplification control. The PCR was performed using an inhibitor-resistant master mix (i.e., Platinum Direct PCR Universal Master Mix). The PCR amplification results were visualized using the HybriDetect 2T lateral flow kit (Milenia Biotec GmbH, Germany) following the manufacturer's instructions.

Similarly, red snapper assay targeting a species-specific SNP was performed using the rhPCR primer-pair (SEQ ID NO: 5) and (SEQ ID NO: 6). A universal primer pair targeting the fish 16S rRNA gene sequence was coamplified in the rhPCR reaction and acted as an IAC. The PCR reaction was performed with RNase H2 enzyme diluted in RNase H2 enzyme buffer, and KAPA Force Master Mix. The reaction was performed on a conventional PCR machine. The PCR amplification profile consisted of an initial denaturation step at 98 °C for 300 s, followed by 35 cycles of denaturation at 95 °C for 10 s, annealing at 58 °C for 30 s, extension at 72 °C for 20 s, and a final extension at 72 °C for 5 min.

### Example 2: Rapid Rnase H-Dependent PCR Lateral Flow Assay for the Detection of Red Snapper

Red snapper (Lutjanus campechanus or Lutjanus purpureus for Caribbean red snapper) is one of the most commercially important and commonly mislabeled seafood species in the United States. Existing molecular methods for its identification are expensive and could take between 3-5 days from shipment to data generation and analysis.

In this study, we have standardized a rapid, rhPCR-coupled lateral flow assay for the identification of red snapper. Overall, the assay consisted of a simple heat-lysis DNA extraction procedure, a duplex rhPCR reaction, a red snapper single nucleotide polymorphism (SNP)-specific rhPCR primer, and a universal internal amplification control (IAC) primer targeting the fish 16S gene sequence. A thermotolerant RNase H2 enzyme was included in the PCRk reaction to activate the red snapper-specific rhPCRk primer. Amplicons generated in the duplex rhPCR reaction were detected using dual target lateral flow strips. The standardized assay was validated with 108 barcoded fish samples from 16 finfish species. Samples identified as Lutjanus campechanus or L. purpureus by DNA barcoding formed three distinct bands, while other fish species formed only two bands on the lateral flow strips. A minimum of 1 ng/reaction was needed to obtain a visible band on the lateral flow dip stick. The assay showed 100% specificity and took 90-120 minutes for completion. Our results confirm the ability of PCR-lateral flow assays as an important, economical, reliable, and alternative tool for species authentication in the seafood industry to mitigate food fraud. The assay generated results that were comparable to DNA barcoding and can be used to authenticate red snapper at various levels of seafood processing and commerce.

### Materials and Methods

### Sample Collections:

A total of 108 fish filet samples spread over 16 commonly retailed species were collected from commercial wholesale outlets across the state of Florida. They consisted of black grouper (n=5), black drum (n=3), blue catfish (n=2), gag grouper (n=2), lane snapper (n=4), red grouper (n=7), red snapper (n=50), rose/pacific lane snapper (n=7), scamp grouper (n=2), silk snapper (n=3), snowy grouper (n=4), tilapia (n=3), vermillion/B-liner snapper (n=6) yellowfin tuna (n=3), yellowtail snapper (n=3), yellow edge grouper (n=4). About 5 g of fish tissue sample was placed into a 2 mL cryotubes and preserved with 1 mL of DNA/RNA Shield™ (Zymo Research, Irvine, CA, USA). The shielded tissue samples were stored at −20 °C for long-term preservation.

### DNA Extraction and Barcoding:

High purity DNA from the fish tissue samples were isolated using DNeasy® Blood & Tissue kit (QIAGEN, Valencia, CA, USA) following manufacturer instructions. The DNA concentration of samples were measured using NanoDrop One spectrophotometer (Thermo Fisher Scientific, Waltham, MA, USA). The DNA samples were diluted to 10 ng/μl and used for testing by the standard DNA barcoding (Handy et al., 2011).

### Rapid Tissue Lysis and Crude DNA Extraction:

Stored tissue samples were defrosted at 4 °C. About 0.5-1 mm of tissue was collected using fine forceps and placed into a microcentrifuge tube. We tested the applicability of Extracta™ DNA Prep for PCR (Quanta Biosciences, Beverly, MA, USA) for rapid isolation of fish tissue DNA. DNA isolation was performed following the manufacturer's recommendations. The DNA concentration was measured using the NanoDrop One spectrophotometer (Thermo Fisher Scientific, Waltham, MA, USA).

### Primer-Design for Lateral Flow PCR Assay:

The COI gene sequences of red snapper, other fish species belonging to the genus Lutjanus, and fish species used for substituting red snapper were aligned to identify the species-specific region and single-nucleotide polymorphism (SNP) markers. Initially, multiple species-specific primer pairs targeting unique red snapper SNP markers were designed using Primer3 software (Untergasser et al., 2012). The amplification potential of these primer pairs was initially tested. Primer-pair showing promising results were modified into rhPCR primer following Integrated DNA Technologies (IDT, Coralville, IA) instructions. First generation rhPCR design (rhPrimer GEN1) containing an RNA base was used (Table 1). The forward red snapper primer was labeled at the 5′ end with FAM, and the reverse primer was labeled with biotin at the 5′ end and used for PCR amplification. To identify any PCR reaction failures, a conserved primer pair targeting fish 16S rRNA gene sequence was used as an internal amplification control (IAC). The forward IAC primer was labeled with FAM, and the reverse primer was labeled with Digoxigenin (DiGN) at the 5′ end (Table 1). The primers were synthesized by IDT (Coralville, Iowa, USA).

### Primer Optimization:

A gradient PCR was used to standardize the annealing temperature of red snapper specific and IAC primer pairs. The gradient PCR was performed on a LightCycler® 96 instrument (Roche Diagnostics Corp., Indianapolis, IN, USA), and the PCR reaction mixture consisted of 5 μl of 2× Apex qPCR GREEN Master Mix (Genesee Scientific, El Cajon, CA, USA), 0.30 μM of forward and reverse rhPCR primers and 20 ng/μl of extracted DNA. The qPCR amplification profile consisted of an initial denaturation step at 95 °C for 900 s followed by 35 cycles of denaturation at 95 °C for 30 s, annealing between 55-62 °C for 30 s and extension at 72 °C for 5 s. A melt curve step was added after the completion of 35 amplification cycles, which consisted of 95 °C for 10 s, 65 °C for 60 s, and 97 °C for 1 s. The real-time PCR results were analyzed using the LightCycler® 96 SW 1.1 software (Roche Diagnostics Corp., Indianapolis, IN, USA). Annealing temperature showing reproducible results and specific melting peaks was used for the rhPCR assay.

### Gel Visualization:

PCR products were confirmed using 1.5% agarose gel (VWR Chemicals, Aurora, Ohio, USA) and a gel electrophoresis apparatus (Benchmark Scientific Inc., Edison, NJ, USA). 10 μl of PCR amplicons and 100 bp DNA marker (New England Biolabs Inc., Ipswich, MA, USA) was loaded into the gel. A voltage supply of 50 mV/cm was selected and allowed to run for 30 minutes. Amplicon bands and ladder were visualized using an ultraviolet (UV) transilluminator light source (Ultra Lum. INC. Paramount, CA, USA). Digital image records were taken using a mobile device.

### PCR Amplification and Optimization:

The PCR amplification potential of the crude DNA extracts was tested with two PCR master mixes, i.e., 2× Kapa Probe Force (ThermoFisher Scientific, Waltham, MA, USA) and Apex 2× RED Taq Master Mix (Genesee Scientific, CA, USA). Crude DNA extracted using Extracta™ DNA Prep for PCR (Quanta Biosciences, Beverly, MA, USA) was diluted (1:2) using nuclease-free water and used for the PCR reaction. Each 20 μl PCR reaction for the two master mixes consisted of 10 μl of PCR master mix, 0.40 μM of forward and reverse rhPCR primers (Rh-315.FAM-RS-430F and Rh-286-Biotin-RS-580R), 0.05 μM of fish IAC primers (318. 16Sar 5′ and 319. 16Sbr-3′), 0.40 μM of RNase H2 enzyme, 2 μl of diluted DNA and 7.15 μl of nuclease-free water. The PCR amplification was carried out using the T 100™ thermal cycler equipment (Bio-Rad Laboratories Inc., Singapore). The PCR amplification profile consisted of an initial denaturation step at 98 °C for 300 s, followed by 35 cycles of denaturation at 95 °C for 10 s, annealing at 58 °C for 30 s, extension at 72 °C for 20 s, and a final extension at 72 °C for 5 min.

### Lateral Flow Detection:

HybriDetect 2T lateral flow kit (Milenia Biotec, GmbH, Germany) was used for the detection of amplified PCR products. Lateral flow assays were performed following the manufacturer's instructions. Briefly, 100 μl of Hybridetect assay buffer (Milenia Biotec, GmbH, Germany) was transferred into a microcentrifuge tube. A 5 μl aliquot of PCR products was added into the assay buffer and vortexed for about 10 s to ensure it was mixed in the buffer. The dipsticks were inserted into the solution and incubated for 5 min. Lateral flow sticks were visually inspected for band formation. Based on the presence or absence of bands, the results were interpreted as positive or negative. Positive species identification was represented by three lines on the strip. In comparison, a negative result was represented by either a single lateral flow control line at the top or two lines, the lateral flow control, and test line A for IAC. Digital image was taken with a mobile device.

### PCR Reaction Analytical Sensitivity:

Red snapper genomic DNA samples were serially diluted from to 0.1 to 500 ng/μl. Diluted DNA samples were used to perform the PCR-lateral flow assay as described above. This was repeated for three different red snapper samples.

### Assay Performance and Statistical Analysis:

The assay sensitivity, specificity, false-positive rate, false-negative rate, positive-predictive value, negative-predictive value, and test accuracy were calculated as previously described (Bosilevac et al., 2019). DNA yield and extraction time were compared using two-way ANOVA (SPSS software version 27) with statistical significance p<0.05. GraphPad Prism version 9.4 was used to plot the graph.

### Results

Assay Standardization: This study aimed to design a simple, species-specific, PCR-lateral flow assay for the onsite authentication of Lutjanus campechanus samples. A standard DNA barcoding method targeting the COI gene sequence was used for the identification of 108 fish samples, which spanned over 16 species (Table 3). The barcoding results served as the basis for the standardization and validation of the red snapper-specific assay.

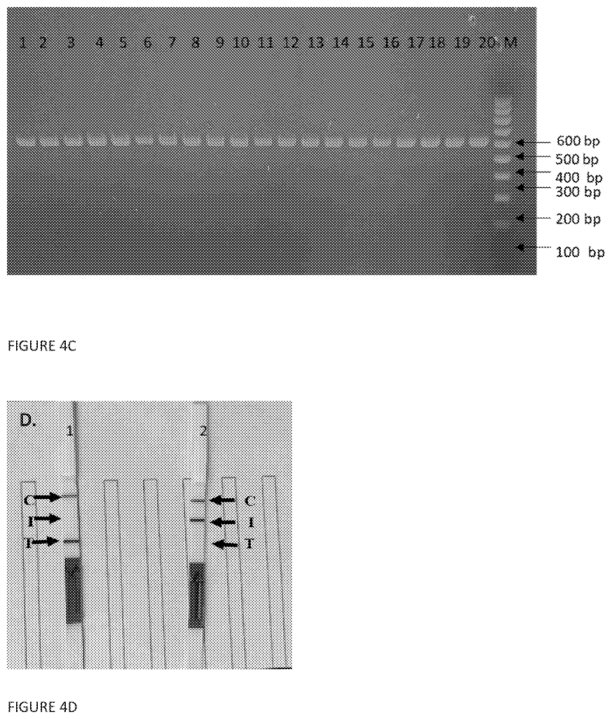

Compared to high-purity DNA isolated using the DNeasy® Blood & Tissue kit, the crude DNA extract isolated using the Extracta DNA PREP for PCR kit generated DNA samples with comparable purity, took shorter sample processing time (~40 mins), generated DNA with significantly higher concentrations (p<0.05), and were found to be suitable for the PCR amplification (FIG. 4A).

Data from the gradient PCR using the RS-430F and RS-580R primer pair showed an optimum annealing temperature of 58 °C. Further, the performance of 2× Kapa Probe Force and Apex 2× RED Taq Master mixes when compared using RS-430F and RS-580R primer pair in a conventional PCR reaction showed superior performance for the 2× Kapa Probe Force master mix with crude DNA extracts. The master mix generated more reproducible results and showed reliable amplification for red snapper samples.

Further, the concentration of the RNase H2 enzyme needed for the activation of blocked rhPrimers was carefully optimized in combination with the 2× Kapa Probe Force master mix. Data from the experiment demonstrated 0.40 mU of enzyme per 20 μl rhPCR reaction was optimum for the specific amplification of red snapper DNA. At this enzyme concentration, all red snapper samples tested positive, whereas other non-target fish DNA samples tested negative.

The applicability of five universal fish PCR primers (Ivanova et al., 2007; Palumbi, 1996) when tested to function as an IAC for the red snapper rhPCR assay showed co-amplification of the universal fish primer, 16Sar-5′ and 16Sbr-3′ in the duplex PCR assay. The primer pair specifically amplified a 617 bp region of the fish 16s rRNA gene of every fish species tested in this study with consistent band intensity and was found to be best suited to serve as an IAC (FIG. 4C). Whereas the other candidates for IAC primer pair failed to amplify (i.e., Mifish-U-F& Mifish-U-R) or generated a non-specific amplification for the red snapper DNA (i.e., FishU-F & FishU-R, dgHCO2198 & m1CO1intF, FF2d & FR1d).

The primer pairs of the standardized duplex assay were modified by the addition of FAM/Biotin and FAM/DiGN modifications to the CO1 and 16s rRNA primers respectively, which enabled the detection of the PCR products using the Hybridetect 2T universal lateral flow test strip. The target amplicons generated a band at the test line (T), while the IAC amplicons were detected at the test line (I) (FIG. 4D).

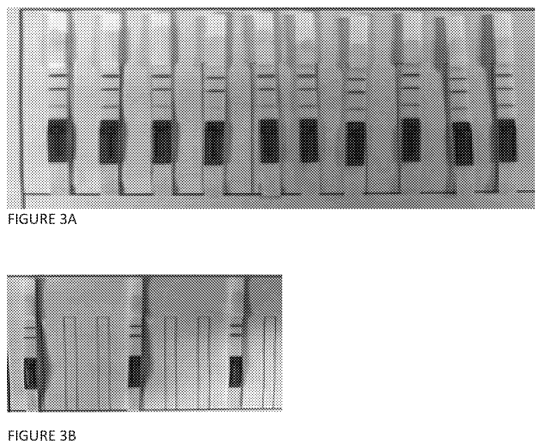

Assay Validation with Red Snapper and Closely Related Species: All 47 red snapper samples (Table 3) tested in this study identified as either L. campechanus or L. purpureus tested positive test and generated three bands on the lateral flow dipstick (FIG. 2). The specificity of assay when tested using DNA from five commonly traded and closely related snapper species (i.e., Lutjanus guttatus, Lutjanus synagris, Lutjanus vivanus, Ocyurus chrysurus, and Rhomboplites aurorubens) generated only two bands (i.e., IAC and lateral flow control) on the lateral flow test strip and test results were interpreted as negative.

Assay Specificity Validation with Non-related Fish Species and PCR Reaction Analytical Sensitivity: Similarly, when the assay specificity was validated with samples belonging to six commonly traded grouper species (i.e., Epinehelus morio, Hyporthodus flavolimbatus, Hyporthodus niveatus, Mycteroperca bonaci, Mycteroperca microlepis and Mycteroperca phenax) generated negative test results with the standardized red snapper PCR lateral-flow assay. Likewise, when the assay was validated with species that are commonly used for substituting red snapper (i.e., Ictalurus furcatus, Pogonias cromis, Thunnus albacares, and Oreochromis spp.) they all tested negative with our assay. The analytical sensitivity of the assay, when tested using a serially diluted DNA sample, were found to be 1 ng/μl. As the DNA concentration increased, it formed a target band of increasing intensity and amplified target DNA till 500 ng/μl concentration.

### DISCUSSION

Red snapper is a premium seafood product that is in high demand throughout the United States. This fish species is sold on the market as headless, and in filleted form. The morphological identification of the red snapper for customers is challenging due to its close similarity with other snapper species. Whereas the seafood industry commonly relies on an internally developed list of key morphological features for the identification of red snapper.

The morphological identification becomes challenging after the fish is processed, which removes the key morphological features. Therefore, to address the seafood industry's need for a rapid, industry-friendly, and low-cost identification method, we have standardized an assay for the onsite identification of red snapper samples.

A simple and quick DNA isolation protocol is pivotal for the development of an industry-friendly, on-site diagnostic method. Multiple commercially available kits were tested, and their applicability was assessed for quickly and efficiently isolating DNA from red snapper tissue samples. Out of the multiple methods tested, the DNeasy Blood & Tissue Kits generated DNA with high purity but took around 120-240 minutes to complete due to parrel extraction of multiple samples and extraction steps. A larger tissue size required a longer lysis time and even required supplementation of proteinase K, making this method suited only for a standard food testing laboratory but not suitable for a rapid extraction as required by seafood processing facilities. In comparison, the Extracta DNA Prep for PCR generated samples with a much higher DNA concentration, took less time to complete (~40 min), was much easier to perform, and showed a 260/280 ratio that was comparable to results obtained using the Dneasy Blood & Tissue Kit. In the past, our laboratory has extensively used and validated the Extracta DNA Prep for PCR for DNA isolation using different matrices (Velez et al. 2022; Kwawukume et al., 2023).

The RS-430F and RS-580R primer pair was one of four conventional PCR primer pairs designed for assay development. The primer pair demonstrated excellent amplification potential but produced a non-specific amplification signal for some species (i.e., red grouper, rose snapper, snowy grouper). To increase the specificity of the assay, the initial PCR primers were converted to rhPCR primers following Gen 1 design guidelines and used in the rhPCR assay. During the assay optimization, the Rnase H2 concentration of 0.40 mU per 20 μl rhPCR reaction was found to be optimum. A further increase in Rnase H2 enzyme concentration resulted in non-specific amplification, and a lower enzyme concertation resulted in the non-activation of the rhPCR primer, resulting in PCR amplification failure.

The rhPCR primer requires sequence-specific hybridization for its activation. Any mismatch during the hybridization step at or near the RNA base significantly reduces the ability of the Rnase H2 enzyme to cleave, minimizing any non-specific amplification. This property of rhPCR is utilized for the standardization of SNP-specific assay. Further, primer-dimer formation is a challenge associated with PCR and lateral flow assays, where the forward and reverse primers bind to each other, generating a false-positive signal (Dobosy et al., 2011). The rhPCR based approach prevents any primer-dimer formation, eliminates any false-positive signals, and increases assay reliability as seen in our results and other previously reported studies (Crissman et al., 2020: Dobosy et al., 2011).

In field conditions, the extracted DNA concentration can vary, thus having a master mix that can generate reproducible results using DNA samples with large variations in concentration (i.e., 1-500 ng) and quality is a critically important feature for an onsite diagnostic assay. There are many PCR mixes that are commercially available for the development of diagnostic assays. However, for the development of an assay suited for the onsite testing of food samples requires a master mix should be robust enough to tolerate a large variation associated with crude DNA extracts. In the past, our laboratory has extensively worked on and assessed the performance of many commercially available PCR mixes on different food matrices (i.e., fresh produce, poultry, red meat, seafood) (Velez et al., 2023; Velez et al., 2022: Kwawukume et al., 2023). Based on our past experiences and the results obtained for red snapper crude DNA extracts, the KAPA probe force mix generated results that were more reproducible. As described by the manufacturer, the master mix is suited for sensitive detection of gene targets even in the presence of inhibitors from blood, tissue, and plant samples without any observable increase in the Cq-values (Roche, 2023: Velez et al., 2022).

The inclusion of an IAC is an essential component of a diagnostic assay (Hoorfar et al., 2004). In field or laboratory conditions, PCR reaction failure may occur due to handling errors or challenging samples, resulting in false-negative test results. Therefore, to further increase the assay's reliability, an IAC was included. A primer pair targeting a conserved 16S rRNA gene sequence was labeled with FAM and DiGN, co-amplified in the PCR reaction, and formed a second band on the two-target lateral-flow strip. Co-amplification of a conventional primer with rhPCR primer in a duplex PCR reaction demonstrates the compatibility of two types of primers in rhPCR reaction.

COI is an appropriate target for the barcoding and identification of animal species due to the low level of genetic divergence of the gene within species and a high level of genetic divergence between species (Hellberg et al., 2017). The species-specific rhPCR primers designed in this study specifically amplified the targeted COI region of only the red snapper samples tested in this study. Three commercially obtained samples that were labeled as red snapper tested negative with our assay and generated only two bands. However, upon further characterization of these three samples using the standard DNA barcoding methods, the samples were identified as Lutjanus vivanus.

The coastal seafood communities coexist and rely on the seafood harvest, processing, sale, and other ancillary support infrastructure. Substitution of seafood species is severely harming the whole seafood community. A set of similar low-cost, species-specific assays focused only on the top five commercially important domestic seafood species can be developed, used to fight species substitution, and have a positive impact on the domestic seafood industry.

### CONCLUSION

This is the first report on the development of a rhPCR-coupled lateral flow assay for species identification. The assay showed 100% specificity for red snapper samples. The assay is best suited for any food testing laboratory or regulatory laboratory and can be combined with a portable PCR and used by stakeholders at any step of commerce, facilitating convenient identification of red snapper samples.

### REFERENCES

1. Bayha, K. M., Graham, W. M., & Hernandez Jr, F. J. (2008). Multiplex assay to identify eggs of three fish species from the northern Gulf of Mexico, using locked nucleic acid Taqman real-time PCR probes. Aquatic Biology, 4(1), 65-73. https://doi.org/10.3354/ab00100
2. Bradley, E., & Bryan, C. E. (1975). Life history and fishery of the Red Snapper (Lutjanus campechanus) in the northwestern Gulf of Mexico 1970-1974.
3. Chou, Q., Russell, M., Birch, D. E., Raymond, J., & Bloch, W. (1992). Prevention of pre-PCR mis-priming and primer dimerization improves low-copy-number amplifications. Nucleic acids research, 20(7), 1717-1723. https://doi.org/10.1093/nar/20.7.1717
4. Crissman, J., Lin, Y., Separa, K., Duquette, M., Cohen, M., Velasquez, C., & Cujec, T. (2020). RNase H-dependent PCR enables highly specific amplification of antibody variable domains from single B-cells. PloS one, 15(11), e0241803. https://doi.org/10.1371/journal.pone.0241803
5. Dawan, J., & Ahn, J. (2022). Application of DNA barcoding for ensuring food safety and quality. Food Science Biotechnology 31, 1355-1364 https://doi.org/10.1007/s10068-022-01143-7
6. Frigerio, J., Gorini, T., Palumbo, C. et al. (2023). A Fast and Simple DNA Mini-barcoding and RPA Assay Coupled with Lateral Flow Assay for Fresh and Canned Mackerel Authentication. Food Anal. Methods 16, 426-435. https://doi.org/10.1007/s12161-022-02429-6
7. Gomes, G., Schneider, H., Vallinoto, M., Santos, S., Orti, G., & Sampaio, I. (2008). Can Lutjanus purpureus (South red snapper) be “legally” considered a red snapper (Lutjanus campechanus)? Genetics and Molecular Biology, 31, 372-376. https://doi.org/10.1590/S1415-47572008000200035
8. Hoorfar, J., Malorny, B., Abdulmawjood, A., Cook, N., Wagner, M., & Fach, P. (2004). Practical considerations in design of internal amplification controls for diagnostic PCR assays. Journal of clinical microbiology, 42(5), 1863-1868. https://doi:10.1128/jcm.42.5.1863-1868.2004
9. https://www.fisheries.noaa.gov/species/red-snapper
10. Hu, Y., Huang, S. Y., Hanner, R., Levin, J. & Lu, X. Study of fish products in metro Vancouver using DNA barcoding methods reveals fraudulent labeling. Food Control 94, 38-47 (2018).
11. Huang, T. S., Marshall, M. R., & Wei, C. I. (1995). Identification of red snapper (Lutjanus campechanus) using electrophoretic techniques. Journal of Food Science, 60(2), 279-283.
12. Isaacs, R. B., & Hellberg, R. S. (2020). Authentication of red snapper (Lutjanus campechanus) fillets using a combination of real-time PCR and DNA barcoding. Food Control, 118, 107375. https://doi.org/10.1016/j.foodcont.2020.107375
13. Ivanova, N. V., Zemlak, T. S., Hanner, R. H., & Hebert, P. D. (2007). Universal primer cocktails for fish DNA barcoding. Molecular Ecology Notes, 7(4), 544-548.
14. Kim, J. H., & Oh, S. W. (2021). A colorimetric lateral flow assay based on multiplex PCR for the rapid detection of viable Escherichia coli O157:H7 and Salmonella Typhimurium without enrichment. LWT; 152, 112242. doi: 10.1016/j.lwt.2021.112242
15. Kwawukume, S., Velez, F. J., Williams, D., Cui, L., & Singh, P. (2023). Rapid PCR-lateral flow assay for the onsite detection of Atlantic White Shrimp. Food Chemistry: Molecular Sciences, 100164.
16. Lee, Y.-M., Lee, S., & Kim, H.-Y. (2021). A Multiplex PCR Assay Combined with Capillary Electrophoresis for the Simultaneous Identification of Atlantic Cod, Pacific Cod, Blue Whiting, Haddock, and Alaska Pollock. Foods, 10(11), 2631. MDPI AG. Retrieved from http://dx.doi.org/10.3390/foods10112631
17. Logan, C. A., Alter, S. E., Haupt, A. J., Tomalty, K., & Palumbi, S. R. (2008). An impediment to consumer choice: overfished species are sold as Pacific red snapper. Biological Conservation, 141(6), 1591-1599.
18. National Marine Fisheries Service (2022). Fisheries of the United States, 2020. U.S. Department of Commerce, NOAA Current Fishery Statistics No. 2020. Available at: https://www.fisheries.noaa.gov/national/sustainable-fisheries/fisheries-united-states
19. Palumbi, S. R. (1996). Nucleic acids II: the polymerase chain reaction. In ‘Molecular Systematics’. (Eds DM Hillis, C. Moritz and BK Mable.) pp. 205-247.
20. https://sequencing.roche.com/us/en/products/group/kapa-probe-force-qpcr-kits.html
21. Shen, K.; Hu, X.; Sun, L.; Han, C.; Yang, J. (2022). Development of a Lateral Flow Strip with a Positive Readout for the Onsite Detection of Aflatoxin B1. Molecules 2022, 27, 4949. https://doi.org/10.3390/molecules27154949
22. Single Laboratory Validated Method for DNA-Barcoding for the Species Identification of Fish | FDA
23. Spencer, E. T., & Bruno, J. F. (2019). Fishy business: red snapper mislabeling along the coastline of the Southeastern United States. Frontiers in Marine Science, 6, 513. https://doi.org/10.3389/fmars.2019.00513
24. Teo, I. A., Choi, J. W., Morlese, J., Taylor, G., & Shaunak, S. (2002). LightCycler qPCR optimisation for low copy number target DNA. Journal of immunological methods, 270(1), 119-133. https://doi.org/10.1016/s0022-1759(02)00218-1
25. Velez, F. J., Bosilevac, J. M., Delannoy, S., Fach, P., Nagpal, R., & Singh, P. (2022). Development and validation of high-resolution melting assays for the detection of potentially virulent strains of Escherichia coli O103 and O121. Food Control, 139, 109095. https://doi.org/10.1016/j.foodcont.2022.109095
26. Velez, F. J., Bosilevac, J. M., Mishra, A., & Singh, P. (2023). Universal hydrolysis probe-based approach for specific detection and genotyping of foodborne pathogens. Journal of Microbiological Methods, 204, 106632. https://doi.org/10.1016/j.mimet.2022.106632
27. Warner, K., Timme, W., Lowell, B., & Hirshfield, M. (2013). Oceana Study Reveals Seafood Fraud Nationwide. Accessed Nov. 9, 2022. Retrieved from https://oceana.org/reports/oceana-study-reveals-seafood-fraud-nationwide/
28. Wells, R. D., Cowan Jr, J. H., & Fry, B. (2008). Feeding ecology of red snapper Lutjanus campechanus in the northern Gulf of Mexico. Marine Ecology Progress Series, 361, 213-225. https://doi.org/10.3354/meps07425
29. Will, S. G. (1999). U.S. Pat. No. 6,001,611. Washington, DC: U.S. Patent and Trademark Office. Accessed from https://patents.google.com/patent/US6001611A/en on Feb. 9, 2023
30. Zhao L, Wang J, Chen M, et al (2022) Development and application of recombinase polymerase amplification assays for rapid detection of Escherichia coli O157 in food. Food Analytical Methods 1-8 https://doi.org/10.1007/s12161-022-02250-1

### Tables

TABLE 1
Primers and Sequences

| Primer | Sequence (5′-3′) |
|---|---|
| WS-197F-FAM | 6-FAM/TAATATAAGCTTCTGA |
| | CTTC (SEQ ID NO: 1) |
| WS-313R-biotin | BiosG/GGCGATACTAGCAGAT |
| | AA (SEQ ID NO: 2) |
| IAC-Shrimp HRM-3F | 6-FAM/GGACGATAAGACCCTA |
| | TAAA (SEQ ID NO: 3) |
| IAC-Shrimp HRM-1R | DiGN/HDTTATATTCYCGTCGC |
| | C (SEQ ID NO: 4) |
| Rh-315.FAM-RS-430F | /56-FAM/GGCCATTAATTTCA |
| | TCACAACrGATCAA/3SpC3/ |
| | (SEQ ID NO: 5) |
| Rh-286-Biotin- | /5BiosG/TGTTTAGATTTCGG |
| RS-580R | TCCGTrGAGAAC/3SpC3/ |
| | (SEQ ID NO: 6) |
| 318.16Sar-5′ | /56-FAM/CGCCTGTTTATCAA |
| | AAACAT |
| | (SEQ ID NO: 7) |
| 319.16Sbr-3′ | /5DiGN/CCGGTCTGAACTCAG |
| | ATCACGT |
| | (SEQ ID NO: 8) |

TABLE 2
Primer table for red
snapper rhPCR lateral flow assay.

| Label | Sequence (5′→3′) | Gene | Amplicon |
|---|---|---|---|
| Rh-315.FAM- | 56-FAM/GGCCATTAA | COI | 170 bp |
| RS-430F: | TTTCATCACAACrGAT | | |
| | CAA/3SpC3/ | | |
| | (SEQ ID NO: 5) | | |
| Rh-286- | /5BiosG/TGTTTAGA | | |
| Biotin- | TTTCGGTCCGTrGAGA | | |
| RS-580R | AC/3SpC3/ | | |
| | (SEQ ID NO: 6) | | |
| 318.16Sar-5′: | /56-FAM/CGCCTGTT | 16S rRNA | 617 bp |
| | TATCAAAAACAT | | |
| | (SEQ ID NO: 7) | | |
| 319.16Sbr-3′: | /5DiGN/CCGGTCTGA | | |
| | ACTCAGATCACGT | | |
| | (SEQ ID NO: 8) | | |

TABLE 3
Results for red snapper specific rhPCR lateral flow assay.

| | | DNA | | | |
|---|---|---|---|---|---|
| Sample | Sample | barcoding/BOLD | | Sample | Sample |
| no. | labelling | identification | Amplicon A | no. | labelling |
| Negative Control DNA | | | | | |
| barcoded samples | | | | | |
| 1 | Silk snapper | Lutjanus vivanus | Absent | Present | − |
| 2 | Silk snapper | Lutjanus vivanus | Absent | Present | − |
| 3 | Silk snapper | Lutjanus vivanus | Absent | Present | − |
| 4 | Yellowtail | Ocyurus | Absent | Present | − |
| | snapper | chrysurus | | | |
| 5 | Yellowtail | Ocyurus | Absent | Present | − |
| | snapper | chrysurus | | | |
| 6 | Yellowtail | Ocyurus | Absent | Present | − |
| | snapper | chrysurus | | | |
| 7 | Lane snapper | Lutjanus synagris | Absent | Present | − |
| 8 | Lane snapper | Lutjanus synagris | Absent | Present | − |
| 9 | Lane snapper | Lutjanus synagris | Absent | Present | − |
| 10 | Lane snapper | Lutjanus synagris | Absent | Present | − |
| 11 | Vermillion/B- | Rhomboplites | Absent | Present | |
| | liner snapper | aurorubens | | | |
| 12 | Vermillion/B- | Rhomboplites | Absent | Present | − |
| | liner snapper | aurorubens | | | |
| 13 | Vermillion/B- | Rhomboplites | Absent | Present | − |
| | liner snapper | aurorubens | | | |
| 14 | Vermillion/B- | Rhomboplites | Absent | Present | − |
| | liner snapper | aurorubens | | | |
| 15 | Vermillion/B- | Rhomboplites | Absent | Present | − |
| | liner snapper | aurorubens | | | |
| 16 | Vermillion/B- | Rhomboplites | Absent | Present | − |
| | liner snapper | aurorubens | | | |
| 17 | Rose/pacific | Lutjanus guttatus | Absent | Present | − |
| | lane snapper | | | | |
| 18 | Rose/pacific | Lutjanus guttatus | Absent | Present | − |
| | lane snapper | | | | |
| 19 | Rose/pacific | Lutjanus guttatus | Absent | Present | − |
| | lane snapper | | | | |
| 20 | Rose/pacific | Lutjanus guttatus | Absent | Present | − |
| | lane snapper | | | | |
| 21 | Rose/pacific | Lutjanus guttatus | Absent | Present | − |
| | lane snapper | | | | |
| 22 | Rose/pacific | Lutjanus guttatus | Absent | Present | − |
| | lane snapper | | | | |
| 23 | Rose/pacific | Lutjanus guttatus | Absent | Present | − |
| | lane snapper | | | | |
| 24 | Yellow Edge | Hyporthodus | Absent | Present | − |
| | grouper | flavolimbatus | | | |
| 25 | Yellow Edge | Hyporthodus | Absent | Present | − |
| | grouper | flavolimbatus | | | |
| 26 | Yellow Edge | Hyporthodus | Absent | Present | − |
| | grouper | flavolimbatus | | | |
| 27 | Yellow Edge | Hyporthodus | Absent | Present | − |
| | grouper | flavolimbatus | | | |
| 28 | Gag grouper | Mycteroperca | Absent | Present | − |
| | | microlepis | | | |
| 29 | Gag grouper | Mycteroperca | Absent | Present | − |
| | | microlepis | | | |
| 30 | Snowy | Hyporthodus | Absent | Present | − |
| | grouper | niveatus | | | |
| 31 | Snowy | Hyporthodus | Absent | Present | − |
| | grouper | niveatus | | | |
| 32 | Snowy | Hyporthodus | Absent | Present | − |
| | grouper | niveatus | | | |
| 33 | Snowy | Hyporthodus | Absent | Present | − |
| | grouper | niveatus | | | |
| 34 | Scamp | Mycteroperca | Absent | Present | − |
| | grouper | phenax | | | |
| 35 | Scamp | Mycteroperca | Absent | Present | − |
| | grouper | phenax | | | |
| 36 | Black | Mycteroperca | Absent | Present | − |
| | grouper | bonaci | | | |
| 37 | Black | Mycteroperca | Absent | Present | − |
| | grouper | bonaci | | | |
| 38 | Black | Mycteroperca | Absent | Present | − |
| | grouper | bonaci | | | |
| 39 | Black | Mycteroperca | Absent | Present | − |
| | grouper | bonaci | | | |
| 40 | Black | Mycteroperca | Absent | Present | − |
| | grouper | bonaci | | | |
| 41 | Red Grouper | Epinehelus morio | Absent | Present | − |
| 42 | Red Grouper | Epinehelus morio | Absent | Present | − |
| 43 | Red Grouper | Epinehelus morio | Absent | Present | − |
| 44 | Red Grouper | Epinehelus morio | Absent | Present | − |
| 45 | Red Grouper | Epinehelus morio | Absent | Present | − |
| 46 | Red Grouper | Epinehelus morio | Absent | Present | − |
| 47 | Red Grouper | Epinehelus morio | Absent | Present | − |
| 48 | Blue catfish | Ictalurus furcatus | Absent | Present | − |
| 49 | Blue catfish | Ictalurus furcatus | Absent | Present | − |
| 50 | Yellowfin | Thunnus | Absent | Present | − |
| | Tuna | albacares | | | |
| 51 | Yellowfin | Thunnus | Absent | Present | − |
| | Tuna | albacares | | | |
| 52 | Yellowfin | Thunnus | Absent | Present | − |
| | Tuna | albacares | | | |
| 53 | Tilapia | Oreochromis | Absent | Present | − |
| | | species | | | |
| 54 | Tilapia | Oreochromis | Absent | Present | − |
| | | species | | | |
| 55 | Tilapia | Oreochromis | Absent | Present | − |
| | | species | | | |
| 56 | Black drum | Pogonias cromis | Absent | Present | − |
| 57 | Black drum | Pogonias cromis | Absent | Present | − |
| 58 | Black drum | Pogonias cromis | Absent | Present | − |
| 59 | Red Snapper | Lutjanus vivanus | Absent | Present | − |
| 60 | Red Snapper | Lutjanus vivanus | Absent | Present | − |
| 61 | Red Snapper | Lutjanus vivanus | Absent | Present | − |
| Positive Control DNA | | | | | |
| barcoded samples | | | | | |
| 62 | Red snapper | Lutjanus | Present | Present | + |
| | | campechanus/ | | | |
| | | purpureus | | | |
| 63 | Red snapper | Lutjanus | Present | Present | + |
| | | campechanus/ | | | |
| | | purpureus | | | |
| 64 | Red snapper | Lutjanus | Present | Present | + |
| | | campechanus/ | | | |
| | | purpureus | | | |
| 65 | Red snapper | Lutjanus | Present | Present | + |
| | | campechanus | | | |
| 66 | Red snapper | Lutjanus | Present | Present | + |
| | | campechanus | | | |
| 67 | Red snapper | Lutjanus | Present | Present | + |
| | | campechanus | | | |
| 68 | Red snapper | Lutjanus | Present | Present | + |
| | | campechanus | | | |
| 69 | Red snapper | Lutjanus | Present | Present | + |
| | | campechanus | | | |
| 70 | Red snapper | Lutjanus | Present | Present | + |
| | | campechanus | | | |
| 71 | Red snapper | Lutjanus | Present | Present | + |
| | | campechanus | | | |
| 72 | Red snapper | Lutjanus | Present | Present | + |
| | | campechanus | | | |
| 73 | Red snapper | Lutjanus | Present | Present | + |
| | | campechanus | | | |
| 74 | Red snapper | Lutjanus | Present | Present | + |
| | | campechanus | | | |
| 75 | Red snapper | Lutjanus | Present | Present | + |
| | | campechanus | | | |
| 76 | Red snapper | Lutjanus | Present | Present | + |
| | | campechanus | | | |
| 77 | Red snapper | Lutjanus | Present | Present | + |
| | | campechanus | | | |
| 78 | Red snapper | Lutjanus | Present | Present | + |
| | | campechanus | | | |
| 79 | Red snapper | Lutjanus | Present | Present | + |
| | | campechanus | | | |
| 80 | Red snapper | Lutjanus | Present | Present | + |
| | | campechanus | | | |
| 81 | Red snapper | Lutjanus | Present | Present | + |
| | | campechanus | | | |
| 82 | Red snapper | Lutjanus | Present | Present | + |
| | | campechanus | | | |
| 83 | Red snapper | Lutjanus | Present | Present | + |
| | | campechanus | | | |
| 84 | Red snapper | Lutjanus | Present | Present | + |
| | | campechanus | | | |
| 85 | Red snapper | Lutjanus | Present | Present | + |
| | | campechanus | | | |
| 86 | Red snapper | Lutjanus | Present | Present | + |
| | | campechanus | | | |
| 87 | Red snapper | Lutjanus | Present | Present | + |
| | | campechanus | | | |
| 88 | Red snapper | Lutjanus | Present | Present | + |
| | | campechanus | | | |
| 89 | Red snapper | Lutjanus | Present | Present | + |
| | | campechanus | | | |
| 90 | Red snapper | Lutjanus | Present | Present | + |
| | | campechanus | | | |
| 91 | Red snapper | Lutjanus | Present | Present | + |
| | | campechanus | | | |
| 92 | Red snapper | Lutjanus | Present | Present | + |
| | | campechanus | | | |
| 93 | Red snapper | Lutjanus | Present | Present | + |
| | | campechanus | | | |
| 94 | Red snapper | Lutjanus | Present | Present | + |
| | | campechanus | | | |
| 95 | Red snapper | Lutjanus | Present | Present | + |
| | | campechanus | | | |
| 96 | Red snapper | Lutjanus | Present | Present | + |
| | | campechanus | | | |
| 97 | Red snapper | Lutjanus | Present | Present | + |
| | | campechanus | | | |
| 98 | Red snapper | Lutjanus | Present | Present | + |
| | | campechanus | | | |
| 99 | Red snapper | Lutjanus | Present | Present | + |
| | | campechanus | | | |
| 100 | Red snapper | Lutjanus | Present | Present | + |
| | | campechanus | | | |
| 101 | Red snapper | Lutjanus | Present | Present | + |
| | | campechanus | | | |
| 102 | Red snapper | Lutjanus | Present | Present | + |
| | | campechanus | | | |
| 103 | Red snapper | Lutjanus | Present | Present | + |
| | | campechanus | | | |
| 104 | Red snapper | Lutjanus | Present | Present | + |
| | | campechanus | | | |
| 105 | Red snapper | Lutjanus | Present | Present | + |
| | | campechanus | | | |
| 106 | Red snapper | Lutjanus | Present | Present | + |
| | | campechanus | | | |
| 107 | Red snapper | Lutjanus | Present | Present | + |
| | | campechanus | | | |
| 108 | Red snapper | Lutjanus | Present | Present | + |
| | | campechanus | | | |

Lastly, it should be understood that while the present disclosure has been provided in detail with respect to certain illustrative and specific aspects thereof, it should not be considered limited to such, as numerous modifications are possible without departing from the broad spirit and scope of the present disclosure as defined in the appended claims.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

1. A method of rapidly determining if a specific food product is present or not, the method comprising:
a. providing a sample comprising at least one target sequence;
b. placing the sample into at least one container;
c. using reagents to amplify the sample;
d. amplifying a sample using a portable amplification device, wherein the sample is amplified by exposing it to different sets of primers and an RNase H2 enzyme in conditions suitable for nucleic acid amplification, wherein each set of primers comprises of a forward and reverse primer, and further wherein at least one set of primers are labelled with FAM/biotin/digoxigenin;
e. detecting whether the specific food product is present or not; and
f. identifying whether the food product is present or not based on the results of step e).

2. The method of claim 1 wherein the food product is beef, pork, poultry, fish, or shellfish or an allergen.

3. The method of claim 2, wherein the sample is Atlantic white shrimp (Litopenaeus setiferus), red snapper (Lutjanus campechanus).

4. The method of claim 1, wherein the food product is a fruit or vegetable or grain or legume.

5. (canceled)

6. (canceled)

7. The method of claim 1, wherein the container further comprises reagents for the specific-amplification of target DNA or target single nucleotide polymorphism (SNP) markers.

8. The method of claim 1, wherein the reagents include:
a. PCR master mix;
b. Taq polymerase; and
c. Primers;

9. The method of claim 8, wherein the primers comprise SEQ ID NO: 1 and SEQ ID NO: 2; or SEQ ID NO: 3 and SEQ ID NO: 4; or SEQ ID NO: 5 and SEQ ID NO: 6; or SEQ ID NO: 7 and SEQ ID NO: 8.

10. The method of claim 8, wherein the Taq polymerase or the master mix has an inhibitor resistant properties.

11. The method of claim 1, wherein the detection means comprises a lateral flow assay or a qPCR machine.

12. (canceled)

13. (canceled)

14. (canceled)

15. The method of claim 8, wherein the reagents are premixed.

16. The method of claim 15, wherein the premixed reagents are activated by PCR grade water or RNase H2 enzyme with reaction buffer.

17-33. (canceled)


SEQUENCE LISTING (8 sequences)

SEQ ID NO: 1 - DNA, 20 bp, synthetic construct
  misc_feature 1..20: note = Description of sequence: Probe
  source 1..20: mol_type = other DNA
  modified_base <1: mod_base = OTHER; note = Tag: 5' 6-FAM (Fluorescein)
taatataagcttctgacttc

SEQ ID NO: 2 - DNA, 18 bp, synthetic construct
  misc_feature 1..18: note = Description of sequence: Probe
  source 1..18: mol_type = other DNA
  modified_base <1: mod_base = OTHER; note = Tag: 5' BiosG
ggcgatactagcagataa

SEQ ID NO: 3 - DNA, 20 bp, synthetic construct
  misc_feature 1..20: note = Description of sequence: Probe
  source 1..20: mol_type = other DNA
  modified_base <1: mod_base = OTHER; note = Tag: 5' 6-FAM (Fluorescein)
ggacgataagaccctataaa

SEQ ID NO: 4 - DNA, 18 bp, synthetic construct
  misc_feature 1..18: note = Description of sequence: Probe
  source 1..18: mol_type = other DNA
  modified_base <1: mod_base = OTHER; note = Tag: DiGN
hdttatattcycgtcgcc

SEQ ID NO: 5 - DNA, 28 bp, synthetic construct
  misc_feature 1..28: note = Description of sequence: Probe
  source 1..28: mol_type = other DNA
  modified_base <1: mod_base = OTHER; note = Tag: 5' 6-FAM (Fluorescein)
  modified_base >28: mod_base = OTHER; note = Modification: 3' Spacer C3
ggccattaatttcatcacaacrgatcaa

SEQ ID NO: 6 - DNA, 26 bp, synthetic construct
  misc_feature 1..26: note = Description of sequence: Probe
  source 1..26: mol_type = other DNA
  modified_base <1: mod_base = OTHER; note = Tag: 5' BiosG
  modified_base >26: mod_base = OTHER; note = Modification: 3' Spacer C3
tgtttagatttcggtccgtrgagaac

SEQ ID NO: 7 - DNA, 20 bp, synthetic construct
  misc_feature 1..20: note = Description of sequence: Probe
  source 1..20: mol_type = other DNA
  modified_base <1: mod_base = OTHER; note = Tag: 5' 6-FAM (Fluorescein)
cgcctgtttatcaaaaacat

SEQ ID NO: 8 - DNA, 22 bp, synthetic construct
  misc_feature 1..22: note = Description of sequence: Probe
  source 1..22: mol_type = other DNA
  modified_base <1: mod_base = OTHER; note = Tag: DiGN
ccggtctgaactcagatcacgt